(19) United States
(12) Patent Application Publication
(10) Pub. No.: US 2005/0054733 A1
(43) Pub. Date: Mar. 10, 2005

(54) Difluoroalkylaromatics

(76) Inventors: Petra Hilgers, Rosrath (DE); Albrecht Marhold, Leverkusen (DE)

Correspondence Address: LANXESS CORPORATION;PATENT DEPARTMENT/ BLDG 14, 100 BAYER ROAD, PITTSBURGH, PA, 15205-9741, US

(21) Appl. No.: 10/935,575

(22) Filed: Sep. 7, 2004

(30) Foreign Application Priority Data
Sep. 9, 2003 (DE) 10341534.3

(51) Int. Cl. A61K 31/137; A61K 31/045; C07C 33/46

(52) U.S. Cl. 514/649; 514/730; 564/386; 568/812

(57) ABSTRACT

The present invention relates to 3,4-difluoro-2-alkylaromatics and 2,4-difluoro-3-alkylaromatics, to a process for their preparation and to their use for preparing active ingredients.

### BACKGROUND OF THE INVENTION

1. Field of the Invention

The present invention relates to 3,4-difluoro-2-alkylaromatics and 2,4-difluoro-3-alkylaromatics, to a process for their preparation and to their use for preparing active ingredients.

2. Brief Description of the Prior Art

2,3,4-Trisubstituted aromatics, especially those which bear fluorine or fluorinated groups as substituents, are important building blocks for active ingredients in pharmaceuticals, in particular in active analgesic and antibacterial ingredients, and crop protection agents, in particular in herbicides and fungicides (see also EP-A 864 559, EP-A 609 798, WO 95/31446, EP-A 625 505, DE-A 36 157 67 and WO 98/46608).

It is therefore desirable to provide 2,3,4-trisubstituted aromatics by which the lipophilicity of the entire active ingredient molecule and thus its effectiveness can be positively influenced and which can be used particularly widely as a synthetic building block.

In addition, it is desirable to provide a process by which these 2,3,4-trisubstituted aromatics can be obtained in good yields starting from readily available reactants.

For example, 2,4-difluoro-3-methylbenzoic acid is disclosed by WO 99/14214 and WO 99/35117. The preparation comprises first the reaction of 2,4-difluorobrombenzene with a strong base and a methylating reagent to give 2,6-difluoro-3-bromotoluene, and also the conversion to an organometallic reagent and subsequent treatment with carbon dioxide.

According to DE-A 36 157 67, 2,4-difluoro-3-methylbenzoic acid is obtained by oxidizing 2,4-difluoro-3-methylacetophenone with bromine and sodium hydroxide solution.

The preparation of 3,4-difluoro-2-methylbenzoic acid is disclosed, for example, by EP-A 609 798, WO 95/31446 and EP-A 625 505 and is effected by dilithiating 3,4-difluorbenzoic acid and subsequently reacting with methyl iodide.

A disadvantage of the processes mentioned is the fact that the repeated use of organometallic reagents on the industrial scale is critical for safety reasons and very expensive overall, and the processes mentioned are different for each product.

### SUMMARY OF THE INVENTION

Compounds of the formula (I) have now been found 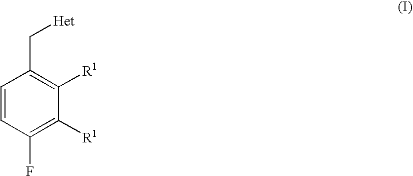
in which
Het is amino or hydroxyl and one of the R¹ radicals is fluorine and the other R¹ radical is C₁-C₄-alkyl.

### DETAILED DESCRIPTION OF THE INVENTION

Preferred compounds of the formula (I) are:
2,3-difluoro-4-methylbenzyl alcohol, 2,4-difluoro-3-methylbenzyl alcohol, 2,3-difluoro-4-methylbenzylamine and 2,4-difluoro-3-methylbenzylamine.

Preference is given to preparing the compounds of the formula (I) in such a way that,
in a step A), compounds of the formula (II) 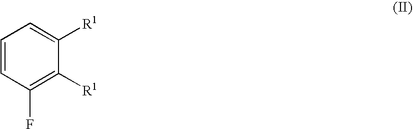
   are converted in the presence of bromine and a catalyst to compounds of the formula (III) 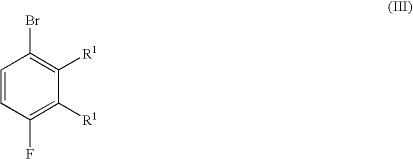
   and, in the case that Het is amino, the compounds of the formula (III),
in a step B1, are reacted with cyanide initially to give compounds of the formula (IV) 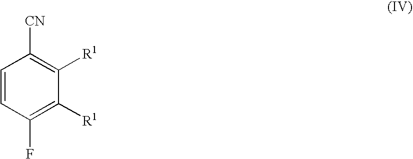
   and subsequently,
in a step B2, converted by reduction to the corresponding compounds of the formula (I), or,
   in the case that Het is hydroxyl,
in a step B3, the compounds of the formula (III) are converted by conversion to an organomagnesium compound, reaction of this organomagnesium compound with carbon dioxide and subsequent treatment with acid to give compounds of the formula (V) 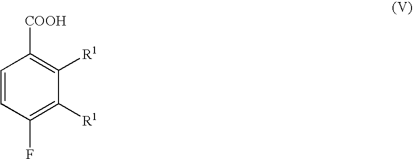
   and the compounds of the formula (V),
in a step B4, are converted using a halogenating agent to compounds of the formula (VI) 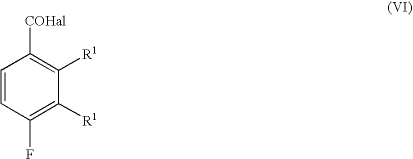
   in which Hal is chlorine or bromine
and, in a step B5, the compounds of the formula (VI) are converted by reduction to the corresponding compounds of the formula (I).

Alternatively to steps B3 to B5, it is also possible that,
in a step B6, the compounds of the formula (III) are converted to an organomagnesium compound, this organomagnesium compound is reacted with an N,N-substituted formamide and subsequently treated with acid to give compounds of the formula (VII) 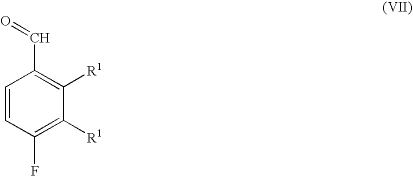
   and then,
in a step B7, the compounds of the formula (VII) are converted by reduction to the corresponding compounds of the formula (I).

In the context of the invention, all radical definitions, parameters and illustrations above and listed hereinbelow, specified in general or within areas of preference, i.e. the particular areas and areas of preference, may be combined as desired.

C₁-C₄-Alkyl is in each case independently a straight-chain, cyclic, branched or unbranched alkyl radical, preferably methyl, ethyl, n-propyl, isopropyl, n-butyl and tert-butyl.

Also encompassed by the invention as indispensable intermediates are:

Compounds of the formula (III) with the exception of 3-bromo-2,6-difluorotoluene.

Compounds of the formula (IV).

Compounds of the formula (V) with the exception of 2,4-difluoro-3-methylbenzoic acid and 3,4-difluoro-2-methylbenzoic acid.

Compounds of the formula (VI) with the exception of 2,4-difluoro-3-methylbenzoyl chloride.

Compounds of the formula (VII).

A preferred compound of the formula (III) is 2-bromo-5,6-difluorotoluene.

Preferred compounds of the formula (IV) are 3,4-difluoro-2-methylbenzonitrile and 2,4-difluoro-3-methylbenzonitrile.

A preferred compound of the formula (VI) is 3,4-difluoro-2-methylbenzoyl chloride.

Preferred compounds of the formula (VII) are 3,4-difluoro-2-methylbenzaldehyde and 2,4-difluoro-3-methylbenzaldehyde.

The bromination in step A) is effected with bromine and in the presence of catalyst, and the catalyst used is preferably iron and iron sulphide. The reaction may be carried out, for example, in a temperature range of −10 to 50° C., preferably at 0 to 30° C.

The reaction in step B1 with cyanide is preferably carried out in such a way that the compounds of the formula (III) are reacted with a metal cyanide, preferably alkali metal and/or copper cyanide, in an aprotic-polar solvent in a temperature range of, for example, 50 to 200° C., preferably of 130 to 170° C.

Aprotic-polar solvents are, for example, nitriles such as acetonitrile, propionitrile or benzonitrile; amides such as N,N-dimethylformamide, N,N-dimethylacetamide, N-methylformanilide, N-methylpyrrolidone or N,N-dimethylimidazolin-2-one, sulphoxides such as dimethyl sulphoxide, sulphones such as tetramethylenesulphone or mixtures of the solvents mentioned.

The reduction in step B2 can preferably be effected by reacting the compounds of the formula (IV) with complex aluminium-hydrogen compounds in an inert solvent at a reaction temperature of, for example, −30 to 70° C., preferably between 0 and 30° C.

Complex aluminium-hydrogen compounds are, for example, those of the formula (VIII)
Met[AlH_{q}R²_{(4-q)}]ₚ (VIII)
in which
Met is a mono- or divalent metal, preferably zinc, lithium, sodium or potassium, and
R² is C₁-C₈-alkyl and
q is 1, 2, 3 or 4, preferably 4 or 1 and
p is the valency of Met.

Particular preference is given to lithium aluminium hydride.

Inert solvents are, for example, aromatic or aliphatic hydrocarbons such as toluene, xylene, various petroleum ethers, hexane, cyclohexane and ethers such as diethyl ether, methyl tert-butyl ether, diisopropyl ether, dioxane, tetrahydrofuran or ethylene glycol dimethyl ether or ethylene glycol diethyl ether or mixtures of such organic solvents.

A possible alternative is catalytic hydrogenation in a protic solvent mixture, in which case suitable catalysts are in particular those which contain palladium, rhodium, iridium or platinum, preference being given to platinum. A particularly preferred catalyst is palladium on carbon with a content of 0.1 to 20% by weight.

Protic solvents are, for example, alcohols such as methanol, ethanol, n- or isopropanol, ethylene glycol monomethyl ether, ethylene glycol monoethyl ether, diethylene glycol monomethyl ether, diethylene glycol monoethyl ether or mixtures thereof.

The reaction in step B3 can preferably be effected by reacting the compounds of the formula (III) with magnesium or Grignard reagents, for example isopropylmagnesium bromide, in an inert solvent as defined above in a temperature range of −20 to 70° C., preferably at 0 to 30° C.

The subsequent reaction with carbon dioxide may be effected by introducing gaseous carbon dioxide or by reacting with solid carbon dioxide. The subsequent acidic workup may be effected, for example, in a manner known per se by acidifying with an aqueous acid and extraction with an organic solvent.

The reaction in B6 can be carried out in a similar manner. In this case, a preferred N,N-substituted formamide is N,N-dimethylformnamide.

The corresponding benzoyl chloride is prepared by reacting the benzoic acid with a chlorinating agent, for example thionyl chloride, in a nonpolar-aprotic solvent, for example by dichloromethane, and a catalytic amount of dimethylformamide in a temperature range of 10-50° C., preferably at 20-25° C.

The conversion to compounds of the formula (VI) in step B4 can preferably be effected by reacting the compounds of the formula (V) with thionyl chloride, thionyl bromide, phosphorus oxychloride, phosphoryl chloride or phosgene.

The reduction in step B5 can preferably be effected by reacting the compounds of the formula (VI) with complex elemental hydrogen compounds in an inert solvent as defined above at a reaction temperature of, for example, −30 to 70° C., preferably between 0 and 30° C.

Complex elemental hydrogen compounds are, for example, those of the formula (IX)
Met[EH_{q}R³_{(4-q)}]ₚ (IX)
in which
Met is a mono- or divalent metal, preferably zinc, lithium, sodium or potassium, and
E is aluminum or boron
R³ is C₁-C₈-alkyl and
q is 1, 2, 3 or 4, preferably 4 or 1 and
p is the valency of Met.

Particular preference is given to sodium borohydride.

The reduction in step B7 can be carried out in a similar manner.

In the inventive manner, the compounds of the formula (I) are obtained in good yields.

The compounds of the formula (I) according to the invention are especially suitable as building blocks for active ingredients in pharmaceuticals, in particular in active analgesic and antibacterial ingredients, and crop protection agents, in particular in herbicides and fungicides, or intermediates thereof.

The invention is further described by the following illustrative but non-limiting examples

### EXAMPLES

### Example 1

### Preparation of 2-bromo-5,6-difluorotoluene

300 g (2.34 mol) of 2,3-difluorotoluene and 7.9 g (141 mmol) of iron were initially charged and 374 g (2.34 mol) of bromine were slowly added dropwise within 4 hours, in the course of which the temperature was kept below 30° C. by cooling. The mixture was left to stir at room temperature overnight (8 hours). The reaction mixture was diluted with 300 ml of water and 300 ml of methyl tert-butyl ether. The phases were separated and the aqueous phase was extracted throroughly with methyl tert-butyl ether. The combined organic phases were washed with sodium thiosulphate solution and water, dried and concentrated. Distillation through a Vigreux column afforded 293 g of colourless liquid; b.p.=68-70° C. at 57 mbar.

### Example 2

### Preparation of 3,4-difluoro-2-methylbenzoic Acid

26.2 g (255 mmol) of a 2 M solution of 2-propylmagnesium chloride in THF were added dropwise under nitrogen and with cooling to a solution of 35 g (170 mmol) of 2-bromo-5,6-difluorotoluene from Example 1 in 80 ml of THF. On completion of addition, the mixture was stirred at room temperature for 15 hours. Carbon dioxide was then introduced slowly over 5 hours and the mixture was stirred for a further 1 hour. For workup, water was slowly added dropwise and the solution acidified to pH 1 using concentrated hydrochloric acid. The aqueous phase was extracted repeatedly with methyl tert-butyl ether and ethyl acetate. The combined organic phases were washed with saturated sodium chloride solution, dried over magnesium sulphate and concentrated. The solid residue was recrystallized from dichloromethane.

21 g of colourless solid were obtained; m.p.: 135° C.

### Example 3

### Preparation of 3,4-difluoro-2-methylbenzoyl Chloride

32.0 g (185 mmol) of 3,4-difluoro-2-methylbenzoic acid from Example 2 were initially charged under nitrogen in 350 ml of dichloromethane and 36.8 g (309 mmol) of thionyl chloride were added dropwise within 5 minutes. The mixture was heated to reflux for 27 hours, and thionyl chloride and dichloromethane were then distilled off. Distillation of the residue afforded 30 g of colourless liquid; b.p.: 35° C. at 2.2 mbar.

### Example 4

### Preparation of 3,4-difluoro-2-methylbenzonitrile

54 g (261 mmol) of 2-bromo-5,6-difluorotoluene from Example 1, 28 g (313 mmol) of copper cyanide and 9.9 g (52 mmol) of copper iodide were initially charged under nitrogen in 600 ml of dimethylformamide. The mixture was heated to reflux for 16 hours. The solid which formed was filtered off and washed repeatedly with a little methyl tert-butyl ether. The filtrate was admixed with 400 ml of water and the organic phase removed. The aqueous phase was extracted four times with approx. 200 ml each time of methyl tert-butyl ether. The combined organic phases were washed with water, dried over magnesium sulphate and concentrated. 35.5 g of a yellow solid were obtained; m.p.: 37-38.5° C.

### Example 5

### Preparation of 3,4-difluoro-2-methylbenzaldehyde

1.3 g (53 mmol) of magnesium were initially charged under nitrogen in 65 ml of tetrahydrofuran and stirred slowly overnight using a magnetic stirrer. The stirrer was switched off and an iodine crystal added to the solution. 10 g (48 mmol) of 2-bromo-5,6-difluorotoluene from Example 2 was then added dropwise at such a rate that continuous gentle reflux was obtained. On completion of addition, stirring was continued until a clear solution was obtained (approx. 2 hours). 7.1 g (97 mmol) of dimethylformamide in 35 ml of tetrahydrofuran were then added dropwise at room temperature within 100 minutes. The reaction mixture was heated to reflux for 4 hours, then hydrolysed by pouring onto ice-water. The remaining magnesium turnings were filtered off. The suspension was acidified using concentrated hydrochloric acid (pH 1). The aqueous phase was extracted 3 times with 50 ml each time of ethyl acetate. The combined organic phases were washed with 100 ml of saturated sodium chloride solution, dried and concentrated.

13 g of a colourless oil were obtained; b.p.: 40° C. (0.9 mbar).

### Example 6

Preparation of 3,4-difluoro-2-methylbenzylamine

33 g (213 mmol) of 3,4-difluoro-2-methylbenzonitrile from Example 5 were taken up under nitrogen in 300 ml of tetrahydrofuran. 8.1 g (213 mmol) of lithium aluminium hydride were added in portions to the solution and the mixture was subsequently stirred at room temperature for 6 hours. Excess lithium aluminium hydride was hydrolysed by cautiously adding water and the precipitate which formed was filtered off. The resulting filtrate was washed with water and the combined aqueous phases were extracted with methyl tert-butyl ether. The combined organic phases were washed with saturated sodium chloride solution, dried over magnesium sulphate and concentrated.

Distillation afforded 17 g of dark red resin; b.p.: 65-70° C. at 2.3-1.8 mbar.

### Example 7

### Preparation of 3,4-difluoro-2-methylbenzyl Alcohol

12.6 g (66 mmol) of 3,4-difluoro-2-methylbenzoyl chloride from Example 3 were initially charged under nitrogen in a mixture of 40 ml of dimethylformamide and 40 ml of tetrahydrofuran. 5 g (132 mmol) of sodium borohydride were added in portions and the mixture was subsequently left to stir at room temperature for 2 hours. The reaction mixture was admixed under ice cooling with 100 ml of ice-water and extracted three times with 70 ml each time of dichloromethane. The combined organic phases were dried over magnesium sulphate and concentrated. Distillation afforded 9 g of colourless liquid; b.p.: 27° C. at 1.4 mbar.

### Example 8

### Preparation of 3-bromo-2,6-difluorotoluene

300 g (2.34 mol) of 2,6-difluorotoluene and 7.9 g (141 mmol) of iron were initially charged and 374 g (2.34 mol) of bromine were slowly added dropwise within 4 hours, in the course of which the temperature was kept below 30° C. by cooling. The mixture was left to stir at room temperature overnight (8 hours). The reaction mixture was diluted with 300 ml of water and 300 ml of dichloromethane. The phases were separated and the aqueous phase was extracted throroughly with dichloromethane. The combined organic phases were washed with sodium thiosulphate solution and water, dried and concentrated. Distillation afforded 450 g of colourless liquid; b.p.=65° C. at 15 mbar.

### Example 9

### Preparation of 2,4-difluoro-3-methylbenzoic Acid

13 g (534 mmol) of magnesium were initially charged under nitrogen in 600 ml of tetrahydrofuran and stirred slowly with a magnetic stirrer overnight. The stirrer was switched off, an iodine crystal was first added, then approx. 10 g of 3-bromo-2,6-difluorotoluene from Example 8 were added dropwise until the reaction commenced. Subsequently, the remaining 90 g of 3-bromo-2,6-difluorotoluene (a total of 483 mmol) were added dropwise with stirring within 65 min, in such a way that continuous gentle reflux was obtained (intrinsic heat). On completion of addition, stirring was continued until a clear solution was obtained. Subsequently, carbon dioxide was introduced for approx. 35 minutes until exothermicity could no longer be observed. 500 ml of water were cautiously added dropwise. The remaining Mg turnings were filtered off and the filtrate was acidified with approx. 250 ml of concentrated hydrochloric acid (pH 1). The solid which precipitated out was filtered off with suction and dried under reduced pressure. Recrystallization from ethanol afforded 38 g of colourless solid; m.p.: 168-170° C.

### Example 10

### Preparation of 2,4-difluoro-3-methylbenzoyl Chloride

25.0 g (145 mmol) of 2,4-difluoro-3-methylbenzoic acid from Example 9 and 2.1 g (29 mmol) of dimethylformamide were initially charged under nitrogen in 300 ml of dichloromethane and 26 g (217 mmol) of thionyl chloride were added dropwise within 5 minutes. The mixture was heated to reflux for 70 hours and then thionyl chloride and dichloromethane were distilled off. 22 g of a colourless solid were obtained.

### Example 11

### Preparation of 2,4-difluoro-3-methylbenzonitrile

100 g (483 mmol) of 3-bromo-2,6-difluorotoluene, 52 g (580 mmol) of copper cyanide and 9.2 g (48 mmol) of copper iodide were initially charged under nitrogen in 700 ml of dimethylformamide. The mixture was heated to reflux for 18 hours. The resulting solid was filtered off and washed repeatedly with little methyl tert-butyl ether. The filtrate was admixed with 400 ml of water and the organic phase was removed. This again formed a precipitate which was filtered off with suction. The aqueous phase was extracted four times with approx. 200 ml each time of methyl tert-butyl ether. The combined organic phases were washed with sodium chloride solution, dried over magnesium sulphate and concentrated. After distillation and recrystallization from ethanol, 49 g of colourless crystals were obtained; m.p.: 40-44° C.

### Example 12

### Preparation of 2,4-difluoro-3-methylbenzaldehyde

1.3 g (53 mmol) of magnesium were initially charged under nitrogen in 20 ml of tetrahydrofuran and stirred slowly overnight using a magnetic stirrer. The stirrer was switched off and an iodine crystal added to the solution. 10 g (48 mmol) of 3-bromo-2,4-difluorotoluene from Example 8 in 15 ml of tetrahydrofuran were then added dropwise at such a rate that continuous gentle reflux was obtained (intrinsic heat). On completion of addition, stirring was continued until a clear solution was obtained (approx. 2 hours). 7.1 g (97 mmol) of dimethylformamide in 35 ml of tetrahydrofuran were then added dropwise at room temperature within 100 minutes. The reaction mixture was heated to reflux for 4 hours, then hydrolysed by pouring onto ice-water. The remaining magnesium turnings were filtered off. The suspension was acidified using concentrated hydrochloric acid (pH 1). The aqueous phase was extracted 3 times with 50 ml each time of ethyl acetate. The combined organic phases were washed with 100 ml of saturated sodium chloride solution, dried and concentrated.

The thus obtained crude product contained 12% of desired target product and higher molecular weight components.

### Example 13

### Preparation of 2,4-difluoro-3-methylbenzylamine

8.8 g (58 mmol) of 2,4-difluoro-3-methylbenzonitrile from Example 11 were taken up under nitrogen in 70 ml of tetrahydrofuran. 4.4 g (116 mmol) of lithium aluminium hydride were added in portions to the solution and the mixture was subsequently stirred at room temperature for 10 hours. Excess lithium aluminium hydride was hydrolysed by cautiously adding 150 ml of water and the precipitate which formed was filtered off. The resulting filtrate was washed with water and the combined organic phases were extracted with methyl tert-butyl ether. The combined organic phases were washed with saturated sodium chloride solution, dried over magnesium sulphate and concentrated.

8.2 g of a colourless liquid were obtained and, according to GC-MS, contained 76% of the target product.

### Example 14

### Preparation of 2,4-difluoro-3-methylbenzyl Alcohol

11 g (58 mmol) of 2,4-difluoro-3-methylbenzoyl chloride from Example 10 were initially charged under nitrogen in a mixture of 80 ml of dimethylformamide and 80 ml of tetrahydrofuran. 4.4 g (115 mmol) of sodium borohydride was added in portions and the mixture was subsequently left to stir at room temperature for 18 hours. The reaction mixture was admixed under ice cooling with 200 ml of ice-water and extracted three times with 100 ml each time of dichloromethane. The combined organic phases were dried over magnesium sulphate and concentrated.

8.5 g of a colourless liquid were obtained and, according to GC-MS, contained 80% of the target product.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. Compounds of the formula (I) 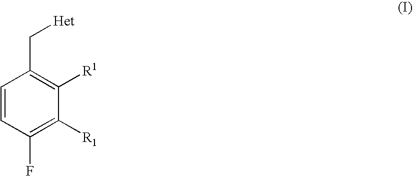
in which
Het is amino or hydroxyl and one of the R¹ radicals is fluorine and the other R¹ radical is C₁-C₄-alkyl.

2. 2,3-Difluoro-4-methylbenzyl alcohol, 2,4-difluoro-3-methylbenzyl alcohol, 2,3-difluoro4-methylbenzylamine and 2,4-difluoro-3-methylbenzylamine.

3. Process for preparing compounds of the formula (I) according to claim 1, comprising:
in a step A), converting compounds of the formula (II) 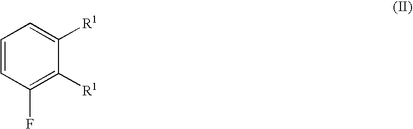
in the presence of bromine and a catalyst to compounds of the formula (III) 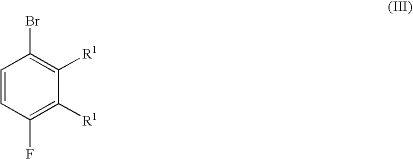
and, in the case that Het is amino,
in a step B1, reacting the compound of formula (III) with cyanide initially to give compounds of the formula (IV) 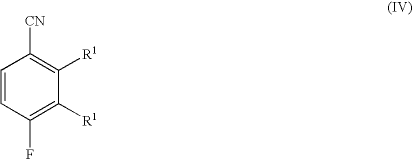
and subsequently,
in a step B2, converting the compound of formula (IV) by reduction to the corresponding compounds of the formula (I), or,
in the case that Het is hydroxyl,
in a step B3, converting the compounds of the formula (III) to an organomagnesium compound, reacting this organomagnesium compound with carbon dioxide and subsequently treating with acid to give compounds of the formula (V) 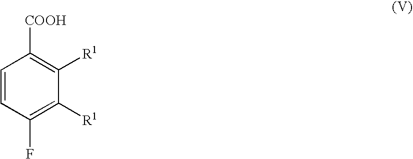
and,
in a step B4, converting the compound of formula (V) using a halogenating agent to compounds of the formula (VI) 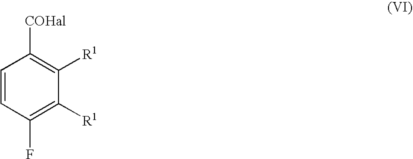
in which Hal is chlorine or bromine
and, in a step B5, converting the compounds of the formula (VI) by reduction to the corresponding compounds of the formula (I).

4. Process according to claim 3, characterized in that, alternatively to the steps B3 to B5,
in a step B6, converting the compounds of the formula (III) to an organomagnesium compound, reacting this organomagnesium compound with an N,N-substituted formamide and subsequently treating with acid to give compounds of the formula (VII) 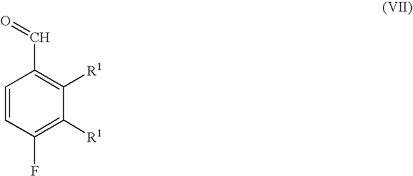
and then,
in a step B7, converting the compounds of the formula (VII) by reduction to the corresponding compounds of the formula (I).

5. Compounds of the formula (III) according to claim 3 with the exception of 3-bromo-2,6-difluorotoluene.

6. Compounds of the formula (IV) according to claim 3.

7. Compounds of the formula (V) according to claim 3 with the exception of 2,4-difluoro-3-methylbenzoic acid and 3,4-difluoro-2-methylbenzoic acid.

8. Compounds of the formula (VI) according to claim 3 with the exception of 2,4-difluoro-3-methylbenzoyl chloride.

9. A process for preparing active ingredients in pharmaceuticals and crop protection agents or intermediates thereof comprising providing compounds according to claim 1 as active ingredients.

10. A process for preparing active ingredients in pharmaceuticals and crop protection agents or intermediates thereof comprising providing compounds according to claim 2 as active ingredients.

11. A process for preparing active ingredients in pharmaceuticals and crop protection agents or intermediates thereof comprising providing compounds according to claim 5 as active ingredients.

12. A process for preparing active ingredients in pharmaceuticals and crop protection agents or intermediates thereof comprising providing compounds according to claim 8 as active ingredients.

13. A process for preparing active ingredients in pharmaceuticals and crop protection agents or intermediates thereof comprising providing compounds which have been prepared according to claim 3 as active ingredients.
